(19) United States
(12) Patent Application Publication
(10) Pub. No.: US 2018/0279938 A1
(43) Pub. Date: Oct. 4, 2018

(54) METHOD OF DIAGNOSING DEMENTIA AND APPARATUS FOR PERFORMING THE SAME

(71) Applicant: GI Signal, Ltd., Busan (KR)

(72) Inventor: Hee Kyong YOO, Seoul (KR)

(73) Assignee: GI Signal, Ltd., Busan (KR)

(21) Appl. No.: 15/786,328

(22) Filed: Oct. 17, 2017

(30) Foreign Application Priority Data
Mar. 28, 2017 (KR) 10-2017-0039368

(51) Int. Cl. A61B 5/00; G06F 19/00

(52) U.S. Cl. CPC A61B 5/4088; G01N 2800/2814; G06F 19/32; A61B 5/0006

(57) ABSTRACT

An apparatus for diagnosing dementia may include database in which a first reference index may be stored. The first reference index may be set based on a standard alpha (α) wave peak levels obtained from EEG measurement signals of a normal person. An alpha wave peak level obtained from EEG measurement signals of a subject may be extracted as a first index. The first index may be compared with the first reference index to diagnose the dementia.

### CROSS-REFERENCES TO RELATED APPLICATION

The present application claims priority under 35 U.S.C. § 119(a) to Korean application number 10-2017-0039368, filed on Mar. 28, 2017, in the Korean Intellectual Property Office, which is incorporated herein by reference in its entirety.

### BACKGROUND

### 1. Technical Field

Various embodiments generally relate to a technology for diagnosing health of human being, more particularly to a method of diagnosing dementia and an apparatus for performing the method.

### 2. Related Art

Technologies for diagnosing health of a human body using bio-signals may have been widely studied.

The bio-signals may include electroencephalogram (EEG), electromyogram (EMG), electrocardiography (ECG), etc.

When a stimulus may be applied to a cerebral cortex, an ionized current may flow through a neuron to form an electric field and a magnetic field. A micro-current change may be measured using an electrode on a scalp to form a waveform. The waveform may correspond to the EEG. The EEG may have about 0 Hz to 100 Hz of frequency band. Because the current change may be dozens of u N, the current change may be amplified. The amplified current change may be recorded as the EEG.

The EEG may be classified into a delta (δ) wave of no more than about 4 Hz, a theta (θ) wave of about 4 Hz to 8 Hz, an alpha (α) wave of about 8 Hz to 12 Hz, a beta (β) wave of about 12 Hz to 30 Hz, and a gamma (γ) wave of about 30 Hz to about 50 Hz in accordance with activation state of a brain, i.e., a vibrated frequency range.

The EEG may be used for diagnosing sleep, awake condition and brain abnormalities. Recently, diagnosis of dementia using the EEG may be widely developed.

The dementia may be a complex clinical syndrome in which perception ability such as memory power, linguistic competence, visual perception, visuospatial formation ability, management ability, etc., may be decreased. Further, the complex clinical syndrome may bring about remarkable inconvenience of personal relations, vocational functions, social life, etc., due to changes of emotion and mentality.

In order to diagnose the dementia, a medical examination by interview may be primarily performed to a subject and family. A perception ability examination may be secondarily performed based on medical examination results. When the subject may require, an EEG test or an MRI may be additionally performed. A light dementia and a light injury of the perception ability may not be early detected using the medical examination. Further, it may be difficult to generalize answers of the subject dependent on subjective feelings.

Therefore, when the dementia may not be progressed in the subject, an aging degree of the brain ability or the dementia may not be early detected only using the medical examination.

Thus, in order to accurately diagnose the dementia, there may exist inconveniences that the subject may actively use medical institutions.

Further, EEG interpretations may be performed by visually recognizing a two-dimensional waveform drawn on an EEG paper, and setting ranges of each of the waveforms. A medical doctor may determine the subject to be normal or abnormal.

A digital EEG measuring instrument such as a brainwave sensor may be developed. However, in order to accurately interpret the waveform, a long skilled observer or a clinical expert may be required. Further, the skilled observers may have different judgment standards.

### SUMMARY

In an embodiment, an apparatus for diagnosing dementia may include database in which a first reference index may be stored. The first reference index may be set based on a standard alpha (α) wave peak levels obtained from EEG measurement signals of a normal person. An alpha wave peak level obtained from EEG measurement signals of a subject may be extracted as a first index. The first index may be compared with the first reference index to diagnose the dementia.

In an embodiment, in a method of diagnosing dementia, a first reference index may be set based on a standard alpha (α) wave peak level obtained from EEG measurement signals of a normal person. An alpha wave peak level obtained from EEG measurement signals of a subject may be extracted as a first index. The first index may be compared with the first reference index to diagnose the dementia.

In an embodiment, an application of a user's terminal may include a function for setting a first reference index based on a standard alpha (α) wave peak level obtained from EEG measurement signals of an normal person, a function for extracting an alpha wave peak level obtained from EEG measurement signals of a subject as a first index, and a function for comparing the first index with the first reference index to diagnose the dementia.

### BRIEF DESCRIPTION OF THE DRAWINGS

FIG. 1 is a block diagram illustrating an apparatus for diagnosing dementia in accordance with example embodiments;

FIG. 2 is a perspective view illustrating an apparatus for measuring an EEG in accordance with example embodiments;

FIG. 3 is a block diagram illustrating an apparatus for diagnosing dementia in accordance with example embodiments;

FIG. 4 is a graph showing EEG data by frequency regions obtained from an EEG of a subject having a normal brain;

FIG. 5 is an absolute power spectrum converted from the graph in FIG. 4;

FIG. 6 is a graph showing EEG data by frequency regions obtained from an EEG of a dementia patient;

FIG. 7 is an absolute power spectrum converted from the graph in FIG. 6; and

FIG. 8 is a flow chart illustrating a method of diagnosing dementia in accordance with example embodiments.

### DETAILED DESCRIPTION

Hereinafter, example embodiments will be described below with reference to the accompanying drawings through various examples of embodiments.

FIG. 1 is a block diagram illustrating an apparatus for diagnosing dementia in accordance with example embodiments.

Referring to FIG. 1, an apparatus 100 for diagnosing dementia in accordance with example embodiments may be configured to receive EEG measurement signals from an EEG measurement apparatus 200.

The dementia diagnosis apparatus 100 may be configured to extract a measurement index of a subject from the EEG measurement signals transmitted from the EEG measurement apparatus 200. In example embodiments, the dementia diagnosis apparatus 100 may extract an absolute power spectrum with respect to a specific EEG, for example, an alpha wave among the EEG measurement signals.

The dementia diagnosis apparatus 100 may set a first reference index based on a peak level of the absolute power spectrum with respect to the at least specific EEG of normal persons having a normal brain. The dementia diagnosis apparatus 100 may compare the first index obtained from the EEG measurement signals of the subject, i.e., the peak level of the absolute power spectrum of the at least specific EEG with the first reference index to diagnose the dementia.

The dementia diagnosis apparatus 100 may set a ratio of absolute power values between the specific EEGs based on the persons having the normal brain as a second reference index. The diagnosing apparatus 100 may compare the second index obtained from the EEG measurement signal of the subject, i.e., the ratio of the absolute power values between the specific EEGs with the second reference index to diagnose the dementia.

According to example embodiments, the dementia diagnosis apparatus 100 may accurately diagnose the dementia based on the reference indexes.

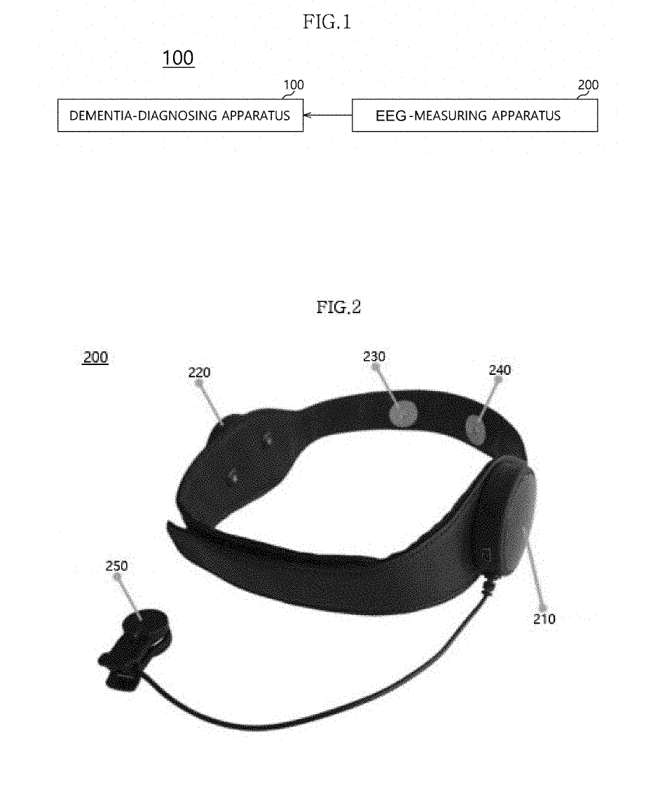

FIG. 2 is a perspective view illustrating an apparatus for measuring an EEG in accordance with example embodiments.

Referring to FIG. 2, an apparatus 200 for measuring an EEG may have a headset worn on a head of the person. Alternatively, the EEG measurement apparatus 200 may have other structures.

The EEG measurement apparatus 200 may include a main frame 210, a power supply 220, a first electrode 230, a second electrode 240 and a reference/ground electrode 250.

The main frame 210 may include a power operation panel, a reset button, etc., of the EEG measurement apparatus 200. The main frame 210 may be configured to control the first electrode 230, the second electrode 240 and the reference/ground electrode 250. The main frame 210 may generate the EEG measurement signals from signals generated from the first electrode 230, the second electrode 240 and the reference/ground electrode 250. The main frame 210 may provide the dementia diagnosis apparatus 100 with the EEG measurement signals.

The power supply 220 may be configured to supply a power to the dementia diagnosis apparatus 100.

The first electrode 230 may be installed at a portion of the head of the subject to measure signals from a left brain. The second electrode 240 may be installed at a portion of the head of the subject to measure signals from a right brain.

A region of the brain in charge of the perception and the learning may relate to a cerebrum cortex neural network. Measuring the signals generated from a frontal lobe of the brain may effectively predict the dementia.

The reference/ground electrode 250 may be worn on an ear of the subject, for example, an earlobe.

In example embodiments, the measurement of the EEGs may be performed under a condition that the eyes of the subject may be closed, i.e., the brain of the subject may relax.

The first and second electrode 230 and 240 may be worn on the portions of the head corresponding to the frontal lobe of the subject. The reference/ground electrode 250 may be worn on the ear of the subject. The EEG measurement apparatus 200 may measure the EEGs of the left and right brains for about five minutes.

The EEG measurement apparatus 200 in FIG. 2 may have a two-channel type. Alternatively, the EEG measurement apparatus 200 may have a four-channel type configured to additionally measure signals from an occipital lobe.

The EEG measurement signals measured by the EEG measurement apparatus 200 may be provided to the dementia diagnosis apparatus 100. The dementia diagnosis apparatus 100 may diagnose the dementia based on the EEG measurement signals.

In example embodiments, the first and second electrodes 230 and 240 may receive ion currents from the cerebrum. The EEG measurement apparatus 200 may amplify a potential difference between the first and second electrodes 230 and 240. The EEG measurement apparatus 200 may filter noises from the amplified voltage to output the EEG measurement signals.

Particularly, the first and second electrodes 230 and 240 may sense the fine current to output signals having very low impedance. The signals outputted from the first and second electrodes 230 and 240 may be applied to a differential amplifier of the main frame 210 without unbalancing of the impedance. The main frame 210 may differentially amplify the signals from the first and second electrodes 230 and 240 with respect to the reference electrode 250 to output the EEG measurement signals by the EEGs.

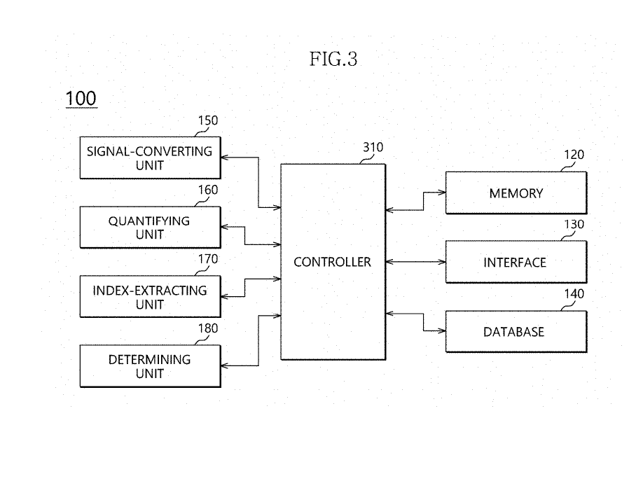

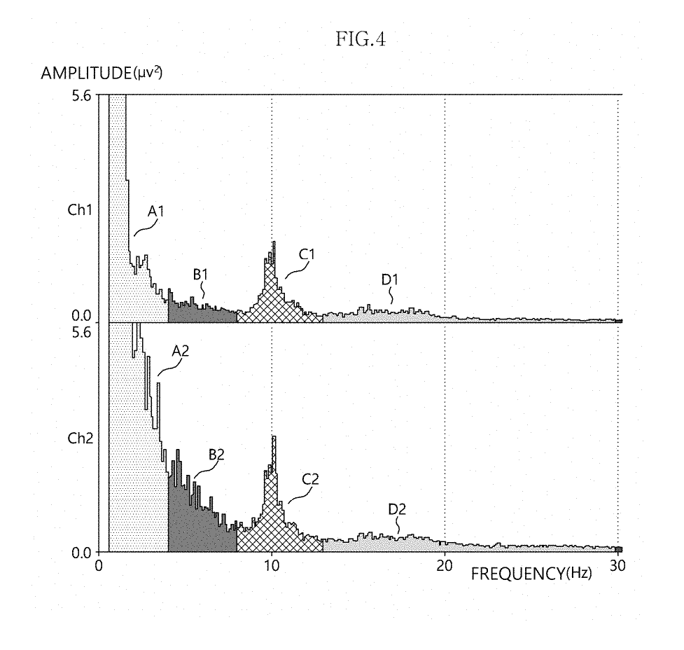

FIG. 3 is a block diagram illustrating an apparatus for diagnosing dementia in accordance with example embodiments.

Referring to FIG. 3, the dementia diagnosis apparatus 100 may include a controller 110, a memory 120, an interface 130, a database 140, a signal-converting unit 150, a quantifying unit 160, an index-extracting unit 170 and a determining unit 180.

The controller 110 may include a central processing unit (CPU). The controller 110 may be configured to control whole operations of the dementia diagnosis apparatus 100.

The memory 120 may be configured to store programs for operating the dementia diagnosis apparatus 100, application programs, control data, operational parameters, processed results, etc.

The interface 130 may configured to provide environments communicated with the EEG measurement apparatus 200 and accessed to the dementia diagnosis apparatus 100 by a user.

In example embodiments, the dementia diagnosis apparatus 100 and the EEG measurement apparatus 200 may be communicated with each other in a wire or wireless communication. The interface 130 may include an interface unit communicated with the EEG measurement apparatus 200.

In order to access the user to the dementia diagnosis apparatus 100, the interface 130 may include an input interface including at least one of a keyboard, a mouse, a touchpad and a microphone, and an output interface including at least one a display and a speaker.

The database 140 may be configured to store the reference index, information of the subject, dementia diagnosis results of the subject, etc.

The signal-converting unit 150 may be configured to convert the EEG measurement signal of the subject as serial data provided from the EEG measurement apparatus 200 into a signal of a frequency region. For example, the signal-converting unit 150 may use a Fast Fourier Transform (FFT).

The EEG measurement signals may be obtained from the left brain Ch 1 of the subject and the right brain Ch2 of the subject. The signal-converting unit 150 may convert the EEG measurement signals from the left brain and the right brain into the signals of the frequency region.

The EEG measurement signals of the left brain and the right brain may be classified into a delta (δ) wave of no more than about 4 Hz, a theta (θ) wave of about 4 Hz to 8 Hz, an alpha (α) wave of about 8 Hz to 12 Hz, a beta (β) wave of about 12 Hz to 30 Hz, and a gamma (γ) wave of about 30 Hz to about 50 Hz by the signal-converting unit 150.

FIG. 4 is a graph showing EEG data by frequency regions obtained from an EEG of a subject having a normal brain. In FIG. 4, a horizontal axis may represent a frequency Hz and a vertical axis may represent an amplitude μ V².

The delta wave A1 and A2, the theta wave B1 and B2, the alpha wave C1 and C2 and the beta wave D1 and D2 with respect to the channels Ch1 and Ch2 may be observed in accordance with the frequencies. The gamma wave may be omitted in FIG. 4.

The quantifying unit 160 may be configured to extract the absolute power spectrum from the signals of the frequency region with respect to the left brain and the right brain converted by the signal-converting unit 150. In example embodiments, the quantifying unit 160 may integrate heights of a graph in the frequency regions with respect to the signals of the frequency regions of the left brain and the right brain converted by the FFT to extract the absolute power spectrum. Thus, the absolute power spectrum may represent the amplitude and the band width of each of the frequencies.

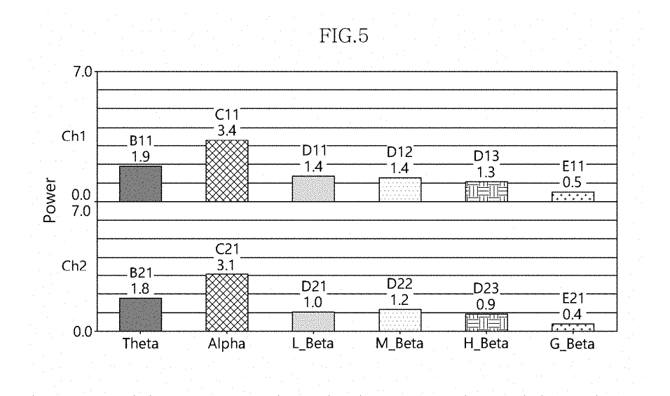

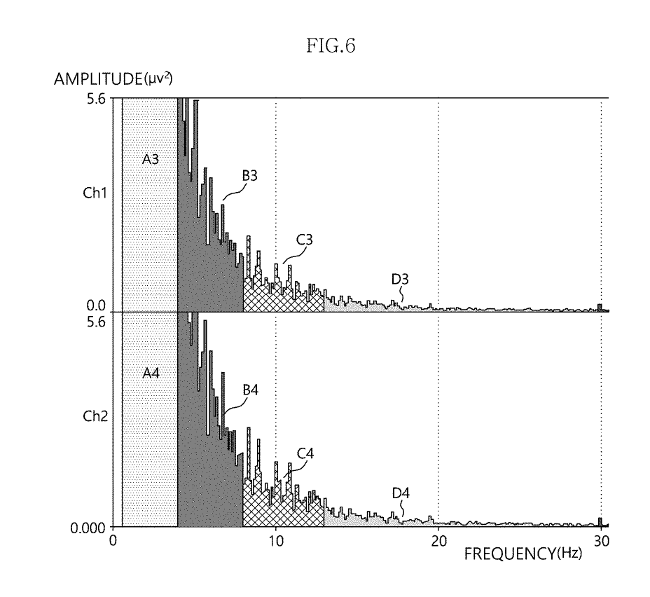

FIG. 5 is an absolute power spectrum converted from the graph in FIG. 4. The absolute power spectrum of the delta wave may be omitted in FIG. 5.

Referring to FIG. 5, absolute power spectrums B11 and B21 of the theta wave B1 and B2, absolute power spectrums C11 and C21 of the alpha wave C1 and C2, absolute power spectrums D11/D12/D13 and D21/D22/D23 subdivided by the delta wave D1 and D2 and absolute power spectrums E11 and E21 of the gamma wave may be shown in FIG. 5.

FIG. 6 is a graph showing EEG data by frequency regions obtained from an EEG of a dementia patient. In FIG. 6, a horizontal axis may represent a frequency Hz and a vertical axis may represent an amplitude μ V².

By converting the serial data into the signals of the frequency regions by the signal-converting unit 150, delta waves A3 and A4, theta waves B3 and B4, alpha waves C3 and C4 and delta waves D3 and D4 with respect to the channels Ch1 and Ch2 by the frequency bands may be observed. The gamma wave may be omitted in FIG. 6.

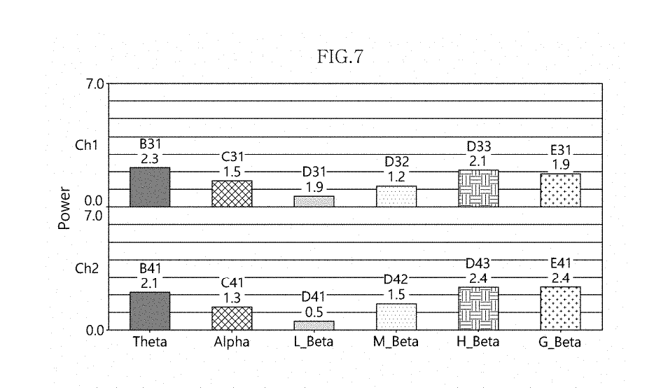

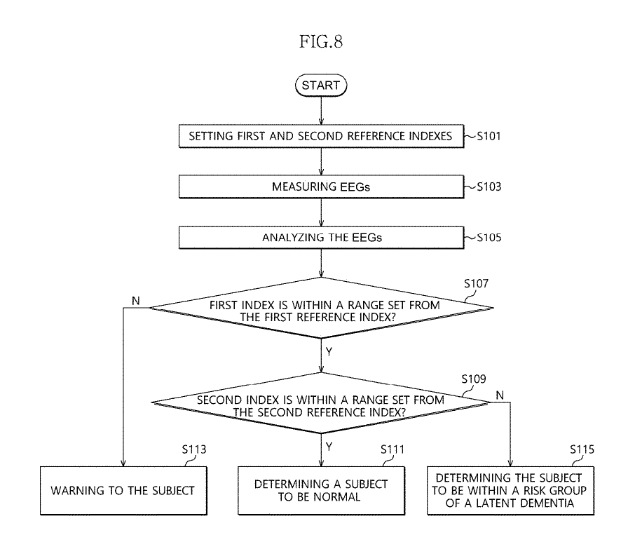

FIG. 7 is an absolute power spectrum converted from the graph in FIG. 6. The absolute power spectrum of the delta wave may be omitted in FIG. 7.

Referring to FIG. 7, absolute power spectrums B31 and B41 of the theta wave B3 and B4, absolute power spectrums C31 and C41 of the alpha wave C3 and C4, absolute power spectrums D31/D32/D33 and D41/D42/D43 subdivided by the delta wave D3 and D4 and absolute power spectrums E31 and E41 of the gamma wave may be shown in FIG. 7.

The index-extracting unit 170 may be configured to extract the first index from the absolute power spectrums obtained by the quantifying unit 160.

As mentioned above, the EEG measurement apparatus 200 may measure the EEG under the condition that the eyes of the subject may be closed. Thus, the alpha wave among the EEG measurement signals may be determined as a principal factor.

First Index=Alpha Wave Peak Level Formula 1

Thus, the peak level with respect to the absolute power spectrum of the alpha wave measured from the subjects having the normal brain, i.e., the alpha wave peak level may be indexed in accordance with a predetermined standard. In example embodiments, the predetermined standard may be determined by ages or generations.

For example, National Reference Standard Center may provide a standard alpha wave peak level by the generations such as following Table 1.

TABLE 1

| Generation | Standard Alpha Peak Level |
|---|---|
| 20 | 9.84 |
| 30 | 9.80 |
| 40 | 9.69 |
| 50 | 9.71 |
| 60 | 9.57 |
| 70 | 9.56 |

However, when a clinical test may be performed, as shown in following Table 2, alpha wave peak levels may be lower than the standards in Table 1.

TABLE 2

| Alpha Wave Peak Level | Standard Alpha Peak | |
|---|---|---|
| of Subjects | Level | Deviation |
| 7.32 | 9.71 | −2.39 |
| 5.61 | 9.57 | −3.96 |
| 5.98 | 9.57 | −3.59 |
| 8.05 | 9.57 | −1.52 |
| 7.75 | 9.56 | −1.81 |

In order to analyze relations between the alpha wave peak levels and the dementia, deviations of the alpha wave peak levels with respect to sixty-two subjects may be analyzed. Analyzed results may be shown in following Table 3.

TABLE 3

| Class | Deviation | Frequency | Percentage |
|---|---|---|---|
| 6 | −2 | 25 | 40% |
| 5 | −1.5 | 14 | 23% |
| 4 | −1 | 16 | 26% |
| 3 | −0.5 | 5 | 8% |
| 2 | 0 | 1 | 2% |
| 1 | 0.5 | 1 | 2% |

Classes 3 to 6 having the deviation of the alpha wave peak levels of about 0 may be no less than about 97% of total dementia patients. Particularly, Classes 5 and 6 may be about 63% of the total dementia patients. Therefore, the alpha wave peak level may be a significant index as a criterion of determining of the dementia or aging of the brain ability.

The values of the alpha wave peak level may represent brain activity that may mean a processing speed of the brain. The processing speed of the brain may be decreased when the value of the alpha wave peak level may be lower than the standard. However, the alpha wave peak level may be within a standard frequency range of about 8 Hz to about 12 Hz of the alpha wave. When the frequency range of the alpha wave of the subject higher than the standard may be overlapped with other frequency range, for example, a frequency range of the beta wave, it may not be determined that the high value of the alpha wave peak level may be good.

Therefore, an offset OFFSET1 may be applied to the standard alpha wave peak level to determine the first reference index. In example embodiments, the offset OFFSET1 may be a value of subtracting a predetermined value, for example, about 15% from the standard alpha wave peak level.

First Reference Index=Standard Alpha Wave Peak Level−OFFSET1 Formula 2

Thus, the index-extracting unit 170 may extract the peak level of the alpha wave among the absolute power spectrums of each of the waves of the subject as the first index.

Additionally, the index-extracting unit 170 may extract the second index from the absolute power spectrums extracted by the quantifying unit 160.

The dementia patient may have the perception ability injury. Therefore, in the dementia patient, the theta wave in a low frequency band may be frequently activated compared than the alpha wave, which may be to be activated in a stable state. Further, as shown in FIG. 4, the alpha wave of the normal person in the frequency region may have a Gaussian distribution. In contrast, as shown in FIG. 6, the alpha wave of the dementia patient may not have the Gaussian distribution.

Referring to FIGS. 4 and 6, in the dementia patient, the peak level of the alpha waves C3 and C4 may be decreased. In contrast, an appearance ratio of the theta waves B3 and B4 may be increased. That is, in the dementia patient, the ratio of the alpha waves C3 and C4 may be relatively higher than the ratio of the theta waves B3 and B4. Further, the alpha waves C3 and C4 may not have the Gaussian distribution.

Therefore, it may be required to set the index to which the amplitudes and the bandwidths of the alpha waves C3 and C4 and the theta waves B3 and B4 may be reflected.

Because the absolute power spectrums extracted by the quantifying unit 160 may reflect the amplitudes and the bandwidths of the theta wave B3 and B4, one index may be set using the absolute power spectrums of the alpha wave and the theta wave among the total absolute power spectrums.

Therefore, the index-extracting unit 170 may extract the absolute power value of the alpha wave with respect to the absolute power value of the theta wave as the second index.

Second Index=Absolute Power Value of Alpha Wave/Absolute Power Value of Theta wave Formula 3

Thus, the absolute power values of the alpha wave and the theta wave measured from the subjects having the normal brain may be indexed in accordance with a predetermined standard. In example embodiments, the predetermined standard may be determined by ages or generations.

For example, National Reference Standard Center may provide the absolute power values of the alpha wave and the theta wave by the generations such as following Table 4.

TABLE 4

| | Absolute | Absolute | |
|---|---|---|---|
| | Power Value of | Power Value of | Second |
| | Standard | Standard | Standard |
| Generation | Theta Wave | Alpha Wave | Index |
| 20 | 2.05 | 3.63 | 1.77 |
| 30 | 2.00 | 3.75 | 1.88 |
| 40 | 2.09 | 3.31 | 1.58 |
| 50 | 2.01 | 3.45 | 1.72 |
| 60 | 1.99 | 3.13 | 1.57 |
| 70 | 2.27 | 3.83 | 1.69 |

In order to analyze relations between the second index and the dementia, deviations of the second index with respect to sixty-two subjects may be analyzed. Analyzed results may be shown in following Table 5.

TABLE 5

| Class | Deviation | Frequency | Percentage |
|---|---|---|---|
| 5 | −1.12 | 6 | 10% |
| 4 | −0.84 | 26 | 42% |
| 3 | −0.56 | 23 | 37% |
| 2 | −0.28 | 6 | 10% |
| 1 | 0 | 1 | 2% |

Classes 3 to 6 having the deviation of the relative power index as the second standard index of about 0 may be no less than about 97% of the total dementia patients. Particularly, Classes 4 and 5 may be about 52% of the total dementia patients. Therefore, the second index may be a significant index as a criterion of determining of the dementia or aging of the brain ability.

When the ratio of the alpha wave may be increased in proportion to increasing of the second standard index, the normal functions of the brain may be maintained. In contrast, when the ratio of the theta wave may be increased, the perception ability of the brain may be decreased. In the dementia patient, the peak level of the alpha wave may be totally decreased and the bandwidth of the alpha wave may also be decreased.

Therefore, an offset OFFSET2 may be applied to the second standard index to determine the second reference index. In example embodiments, the offset OFFSET2 may be a value of subtracting a predetermined value, for example, about 20% from the second standard index.

Second Reference Index=(Absolute Power Value of Standard Alpha Wave/Absolute Power Value of Standard Theta Wave)−OFFSET2 Formula 4

The determining unit 180 may compare the first and second indexes extracted by the index-extracting unit 170 with the first and second reference indexes in the database 140, respectively, to diagnose the dementia.

According to example embodiments, the perception ability of the brain may be objectively measured using the first reference index and the second reference index.

In example embodiments, the first reference index may be regarded as a predominant term under a condition that the lowest two classes may be relatively more screened. The first reference index may be preferentially applied to analysis of the EEG.

After analysis results may be within a range set from the first reference index, examination results may then be within a range set from the second reference index to predict the aging of the brain ability and the latent dementia patient.

FIG. 8 is a flow chart illustrating a method of diagnosing dementia in accordance with example embodiments.

Referring to FIG. 8, in step S101, the reference index may be set.

The reference index may include the first reference index obtained by applying the first offset, for example, about −15% to the peak level of the alpha wave, and the second reference index obtained by applying the second offset, for example, about −20% to the ratio of the absolute power value of the alpha wave with respect to the absolute power value of the theta wave.

After the reference index may be set, in step S103, the EEG measurement apparatus 200 may be worn on the subject. The EEG measurement apparatus 200 may measure the EEG of the subject through the two or four channels during the eyes of the subject may be closed.

In step S105, the EEG measurement signals from the left brain and the right brain measured by the EEG measurement apparatus 200 may be provided to the dementia diagnosis apparatus 100. The dementia diagnosis apparatus 100 may analyze the absolute power spectrums. The dementia diagnosis apparatus 100 may convert the EEG measurement signals into the signals of the frequency regions through the FFT. The dementia diagnosis apparatus 100 may extract the first index and the second index of the subject from the absolute power spectrums.

In step S107, after analyzing the EEG measurement signals, whether the first index as the peak level of the alpha wave of the subject may be within the range of the first reference index or not may be identified.

When the first index may be within the range of the first reference index, in step S109, whether the second index as the ratio of the absolute power value of the alpha wave with respect to the absolute power value of the theta wave may be within the range of the second reference index or not may be identified.

When the second index may be within the rage of the second reference index, in step S111, the brain of the subject may be determined to be normal.

In contrast, when the first index may not be within the range of the first reference index, in step S113, the subject may be determined and warned to be within a risk group of the latent dementia.

When the second index may not be within the rage of the second reference index, in step S115, the subject may receive a cautious warning although not in the risk group of the latent dementia.

The first and second reference indexes may be set by the generations. Thus, in order to analyze the first and second indexes, it may be required to input generation information of the subject into the dementia diagnosis apparatus 100.

According to example embodiments, the brain ability may be diagnosed using the first and second reference indexes and the first and second index of the subject. Therefore, the dementia may be early diagnosed. Further, the latent dementia group may be early screened to control the aging speed of the brain. Furthermore, the aging of the brain ability of each of the persons may be accurately predicted by subdividing the first and second reference indexes.

Moreover, the prediction results may have a level objectively determined by a general person so that the general person may check his own brain without using of medical institutions.

Further, dementia symptom may be early detected based on the prediction results of the aging of the brain ability so that dementia prevention may be useful although observable symptoms may not be founded. When a proper EEG train may be performed in accordance with the diagnosis results, the dementia may be prevented and the aging speed of the brain may also be improved.

The dementia diagnosis method may be installed in an application of a user's terminal. The application may store the reference index. The application may extract the first and second indexes from the EEG measurement signals provided from the EEG measurement apparatus 200 to diagnose the dementia.

In example embodiments, the user's terminal may include a personal computer, a smart phone, a tablet PC, a notebook computer, etc.

The above embodiments of the present disclosure are illustrative and not limitative. Various alternatives and equivalents are possible. The examples of the embodiments are not limited by the embodiments described herein. Nor is the present disclosure limited to any specific type of semiconductor device. Other additions, subtractions, or modifications are obvious in view of the present disclosure and are intended to fall within the scope of the appended claims.

What is claimed is:

1. An apparatus for diagnosing dementia, the apparatus comprising:
a database configured to store a first reference index, which is set based on a peak level of a standard alpha wave obtained from electroencephalogram (EEG) measurement signals of a normal person,
wherein a peak level of an alpha wave obtained from EEG measurement signals of a subject is extracted as a first index, and the first index is compared with the first reference index to diagnose the dementia.

2. The apparatus of claim 1, wherein the first reference index is set by reflecting a first offset to the peak level of the standard alpha wave by generations.

3. The apparatus of claim 1, wherein the database further stores a second reference index set based on a ratio of an absolute power value of the standard alpha wave with respect to an absolute power value of a standard theta wave obtained from the EEG measurement signals of the normal person, a ratio of an absolute power value of the alpha wave with respect to an absolute power value of the theta wave obtained from the EEG measurement signals of the subject is extracted as a second index, and the second index is compared with the second reference index to diagnose the dementia.

4. The apparatus of claim 3, wherein the second reference index is set by reflecting a second offset to the ratio of the absolute power value of the standard alpha wave with respect to the absolute power value of the standard theta wave by generations.

5. The apparatus of claim 1, further comprising:
a signal-converting unit configured to convert the EEG measurement signals of the subject into signals of a frequency region;
a quantifying unit configured to quantify the signals of the frequency region to extract peak levels by the EEGs;
an index-extracting unit configured to extract the peak level of the alpha wave among the peak levels of the EEGs as the first index; and
a determining unit configured to compare the first index with the first reference index corresponding to generations of the subject to diagnose a brain of the subject.

6. The apparatus of claim 5, wherein the database further store a second reference index set based on a ratio of an absolute power value of the standard alpha wave with respect to an absolute power value of a standard theta wave obtained from the EEG measurement signals of the normal person, the quantifying unit further extracts absolute power values of the EEGs, the index-extracting unit further extract a ratio of an absolute power value of the alpha wave with respect to an absoluter power value of a theta wave among the absolute power values by the EEGs as a second index, and the determining compare the second index with the second reference index by the generations in accordance with comparison results between the first index and the first reference index to diagnose the dementia.

7. A method of diagnosing dementia, the method comprising:
setting a first reference index set based on a peak level of a standard alpha wave obtained from electroencephalogram (EEG) measurement signals of a normal person;
is extracting a peak level of an alpha wave obtained from EEG measurement signals of a subject as a first index; and
comparing the first index with the first reference index to diagnose the dementia.

8. The method of claim 7, wherein extracting the first index comprises:
converting the EEG measurement signals of the subject into signals of a frequency region;
quantifying the signals of the frequency region to extract peak levels by the EEGs; and
extracting the peak level of the alpha wave among the peak levels of the EEGs as the first index.

9. The method of claim 7, wherein the first reference index is set by reflecting a first offset to the peak level of the standard alpha wave by generations.

10. The method of claim 7, further comprising:
setting a second reference index set based on a ratio of an absolute power value of the standard alpha wave with respect to an absolute power value of a standard theta wave obtained from the EEG measurement signals of the normal person;
extracting a ratio of an absolute power value of the alpha wave with respect to an absolute power value of the theta wave obtained from the EEG measurement signals of the subject as a second index; and
comparing the second index with the second reference index in accordance with comparison results between the first index and the first reference index to diagnose the dementia.

11. The method of claim 10, wherein extracting the second index comprises:
converting the EEG measurement signals of the subject into signals of a frequency region;
quantifying the signals of the frequency region to extract absolute power values by the EEGs; and
extracting a ratio of the absolute power value of the alpha wave with respect to an absoluter power value of a theta wave among the absoluter power values of the EEGs as the second index.

12. The method of claim 10, wherein the second reference index is set by reflecting a second offset to the ratio of the absolute power value of the standard alpha wave with respect to the absolute power value of the standard theta wave by generations.

13. The method of claim 10, wherein comparing the second index with the second reference index is performed when the first index is within a range set from the first reference index.

14. The method of claim 13, further comprising determining the subject to be normal when the second index is within a range set from the second reference index.

15. The method of claim 13, further comprising warning the subject to be abnormal when the second index is not within a range set from the second reference index.

16. The method of claim 10, further comprising determining the subject to be within a risk group of a latent dementia when the first index is within a range set from the first reference index.
